(19) United States
(12) Patent Application Publication
(10) Pub. No.: US 2001/0023330 A1
(43) Pub. Date: Sep. 20, 2001

(54) Transdermal drug delivery system and method

(76) Inventor: Yoram Palti, Haifa (IL)

Correspondence Address: SHEARMAN & STERLING, INTELLECTUAL PROPERTY DOCKETING, 599 LEXINGTON AVENUE, NEW YORK, NY, 10022, US

(21) Appl. No.: 09/838,105

(22) Filed: Apr. 19, 2001

Related U.S. Application Data
(62) Division of application No. 09/264,325, filed on Mar. 8, 1999.

(51) Int. Cl. A61N 1/30

(52) U.S. Cl. 604/20

(57) ABSTRACT

A transdermal iontophoresis electrode assembly includes one or more arrays of electrodes separated by dividing members. The spacing between the electrodes and/or the size of the electrodes is adjusted for controlling depth of penetration and current density. An alternative electrode assembly includes a housing, an electrode within the housing, a membrane covering an open end of the housing, a drug to be administered disposed within the housing, and an insulating layer covering the open end of the housing. The insulating layer includes apertures for channeling current passed through the electrode. The sizes of the apertures may be adjusted for achieving a desired depth of penetration or current density. In transdermal iontophoresis methods, relational criteria relating i) AC frequency/pulse duration to nerve or muscle tissue stimulation, ii) maximum current density to skin burning, iii) ionic concentration to depth of penetration and AC frequency/pulse duration, and iv) electrode size to current density, are used to determine operational criteria for carrying out transdermal iontophoresis. In a transdermal iontophoresis system the electrical signal used to perform transdermal iontophoresis is an AC signal having a frequency of greater than 100 Hz, or an alternating signal having a pulse duration of less than 2 milliseconds. In a further transdermal iontophoresis system, the ionic concentration of the drug is varied to achieve a desired depth of penetration.

### RELATED APPLICATIONS

This application is a divisional of U.S. patent application Ser. No. 09/264,325, filed on Mar. 8, 1999, entitled “Transdermal Drug Delivery System and Method,” the entire contents of which are incorporated by reference herein.

### BACKGROUND OF THE INVENTION

1. Field of the Invention

The present invention relates generally to the use of transdermal iontophoresis for drug delivery, and more particularly to a system and method for optimally controlling the delivery of the drug by using certain electrode structures and configurations, by varying the electrical characteristics of the electric signal used to carry the drug, and by adjusting the ionic characteristics of the drug.

3. Description of the Related Art

Transdermal iontophoresis is a known but not widely used method for delivering drugs through the skin using electric current. As shown in FIG. 1, a power source 2, which may be controlled by a controller 4, generates an electrical current, which is applied through electrodes 5, 7. The electric current is normally a DC signal generally in the range of 0.1-10 mA. Electrodes 5,7, which are usually constructed of metal, are placed on the skin at a location at which a “drug” has been topically applied. The flow of current from one electrode 5 to the other electrode 7 carries ionized molecules of the drug through skin 10, 12 into the body. Examples of transdermal iontophoresis techniques are shown, for example, in U.S. Pat. Nos. 4,301,794; 4,406,658; 4,786,278; 4,792,702; 5,087,243; 5,135,478; 5,279,543; 5,450,845; 5,618,265; 5,667,487; and 5,730,716, the contents of which are incorporated herein by reference.

Transdermal iontophoresis may be used to administer not only drugs, but any other desired substance for which the technique is applicable, which in general includes any substance that may be ionized or that may be carried in a substance that may be ionized. As used herein, “drug” shall be used to denote any substance, which may be delivered using the transdermal iontophoresis technique, including an ionized drug alone or in combination with a carrier used to transport the drug into the body.

The main theoretical advantages of using transdermal iontophoresis are its ability to replace subcutaneous or intramuscular injection of a drug with a painless topical application of the drug, the ability to control dosage, and the ability to provide continuous or slow delivery of the drug over a period of time. Thus transdermal iontophoresis is an alternative to traditional drug injection using a syringe and the concomitant risks associated with skin puncture and penetration. Despite these advantages, there are also numerous problems associated with transdermal iontophoresis.

One problem results from the use of DC current to deliver the drug, where the control of drug delivery is maintained by adjusting current amplitude. The application of intense current over time may cause physical discomfort such as skin irritation (burning) because the current generates heat, and muscle-nerve stimulation because nerve and muscle fibers have excitability parameters that control their ability to respond to stimulation over time. Also, the current may cause accumulation of irritating substances. As a result, in most cases, high current intensity needed for optimal drug delivery may not be maintained for sufficient duration without resulting in burning and muscle stimulation.

To the extent that efforts have been made to improve transdermal iontophoresis, these efforts have focused piece-meal on the various problems associated with transdermal iontophoresis. None of the related art addresses the complex relationship between DC pulse duration or AC frequency, tissue and muscle stimulation threshold, burning threshold, and electrode geometry that result in these problems. For example, it has been proposed to modify the electrical signal to use an alternating current signal, to pulse the DC current, and/or to periodically reverse the DC current for a short period of time. Examples of these techniques are shown in U.S. Pat. Nos. 5,571,149; 4,301,794; 4,340,047; 5,224,927; 4,792,702; and 5,013,293, the contents of which are incorporated herein by reference. The use of reversing currents in the prior art generally involve reversing of currents over a cycle of long duration, (e.g., U.S. Pat. Nos. 5,571,149; 4,301,794; 4,340,047; 5,224,927; 4,792,702; 5,013,293; and 5,006,108), and do not involve symmetrical polarity such that net flow of current is zero (e.g., U.S. Pat. Nos. 5,135,478; 5,328,452; and 5,499,971). In general, none of these devices addresses the use of reversing current to address the issue of stimulating nerve and muscle fibers beyond the threshold for irritation and burning, nor the issue of the lack of depth penetration that normally results from using a high-frequency AC signal for drug delivery. In addition, none of the prior art addresses the relationship between AC frequency and stimulation power on the basis of a sensitivity curve. While U.S. Pat. No. 5,499,971 discusses the use of certain waveforms, which are generally DC waveforms, to provide better control over cardiac arrhythmia, it fails to address the issue of depth of penetration. Also, while several prior patents (e.g., U.S. Pat. Nos. 4,950,229 and 5,284,471) relate vaguely to depth of penetration using DC currents, these patents address electrode geometry spacing without linking the depth of penetration issue to the problem of tissue stimulation, electrode area, current density, and pulse duration. Other patents (e.g., U.S. Pat. Nos. 4,211,222 and 5,310,403) discuss the use of multiple electrodes, which may be formed into an array, for transdermal iontophoresis, but make no provision for controlling depth of penetration of the drug while overcoming the problems of burning and muscle stimulation.

In general, none of the related art addresses the complex relationship between AC frequency, tissue and muscle stimulation threshold, burning threshold, and electrode geometry that limit the applicability of transdermal iontophoresis. Accordingly, it would be desirable to have a transdermal iontophoresis system that takes into account these various factors and that enables control over depth of penetration, while at the same time reducing or eliminating side effects, such as burning and muscle stimulation, normally associated with transdermal isontophoresis.

### SUMMARY OF THE INVENTION

The present invention overcomes the problems associated with the prior transdermal iontophoresis techniques, including, damage and irritation to the skin and nerve and muscle fibers, and lack of control over drug penetration depth. The system uses an electrical signal having particular electrical characteristics in combination with a drug having particular charge and geometrical distribution characteristics in further combination with specialized electrodes. This combination enables the system of the invention to reduce or eliminate muscle and nerve tissue stimulation and burning, while at the same time enabling control over drug dosage and penetration depth.

A transdermal iontophoresis electrode assembly includes a housing having an array of electrodes disposed within the housing, and a membrane, which covers an aperture on the end of the housing. Dividing members within the housing separate each electrode of the array and form a channel associated with each electrode. Each channel is filled with the drug to be administered, and is electrically insulated from each other channel. An open end of each channel is adjacent to the membrane so that the membrane covers the end of the channel. Each electrode of the array is preferably in electrical communication with each other electrode, although one or more of the electrodes in the array may be separately controllable from the other electrodes in the array. The housing and the dividing members are preferably constructed of a non-electrically conducting material and the array of electrodes is mounted in the housing opposite the aperture. The housing and dividing members may be integrally constructed, if desired.

In a method of using the electrode assembly, the spacing between the electrodes of the electrode array and/or the size of the electrodes are adjusted for achieving a desired depth of penetration or current density. In an alternative method of using the electrode, a set of desired criteria for delivery of the drug is established, with the set of desired criteria selected from the group consisting of muscle and nerve tissue stimulation, depth of penetration, drug delivery rate/magnitude, and permissible maximum current density to avoid burning. Using relational criteria selected from the group of criteria relating i) AC frequency/pulse duration to nerve or muscle tissue stimulation, ii) maximum current density to skin burning, iii) ionic concentration to depth of penetration and AC frequency/pulse duration, and iv) electrode size to current density, transdermal iontophoresis operational criteria are determined for achieving the set of desired criteria, with the operational criteria selected from the group consisting of AC frequency/pulse duration, current density, ionic concentration, electrode size, and electrode spacing. Finally, transdermal iontophoresis is conducted using the transdermal iontophoresis operational criteria.

In an alternative embodiment, an electrode assembly includes first and second arrays of electrodes disposed within a housing, with each electrode of each of the first and second arrays of electrodes being in electrical communication with each other electrode in such array. Dividing members separate each electrode of each of the first and second arrays of electrodes, and form a first channel filled with the drug associated with each electrode of the first electrode array. The dividing members electrically insulate each electrode of each of the first and second electrode arrays and electrically insulate each first channel from the other first channels and from each electrode of the second array of electrodes. Each second electrode is in electrical contact with, and preferably in direct contact with, the membrane. If desired, the second array of electrodes may be molded into the dividing members. In an alternative embodiment, the dividing members form a second channel associated with each electrode of the second array of electrodes, with each second channel also being filled with the drug to be administered. The dividing members electrically insulate each second channel from the first channels and from the other second channels.

In a transdermal iontophoresis method using this type of electrode assembly the sizes of the electrodes and/or the spacing between the electrodes of the first electrode array and the electrodes of the second electrode array are adjusted for achieving a desired depth of penetration or current density.

A further alternative transdermal iontophoresis electrode assembly includes a housing, an electrode within the housing, a membrane covering an open end of the housing, a drug to be administered disposed within the housing in electrical contact with the membrane, and an insulating layer positioned adjacent to the membrane, also covering the open end of the housing. The insulating layer includes a plurality of apertures extending therethrough for channeling current passed through the electrode and the drug within the housing. The housing is preferably constructed of a non-electrically conducting material and the insulating layer is positioned interior of the membrane. In a transdermal iontophoresis method using this electrode assembly, the size of the apertures in the insulating layer is adjusted for achieving a desired depth of penetration or current density.

In a transdermal iontophoresis method a set of desired criteria for delivery of a drug using transdermal iontophoresis is determined, with the set of desired criteria selected from the group consisting of muscle and nerve tissue stimulation, depth of penetration, drug delivery rate/magnitude, and maximum current density to avoid burning. Using relational criteria selected from the group of criteria relating i) AC frequency/pulse duration to nerve or muscle tissue stimulation, ii) maximum current density to skin burning, iii) ionic concentration to depth of penetration and AC frequency/pulse duration, and iv) electrode size to current density, transdermal iontophoresis operational criteria for achieving the set of desired criteria are determined selected from the group consisting of AC frequency/pulse duration, current density, ionic concentration and electrode size. Finally transdermal iontophoresis is carried out using the transdermal iontophoresis operational criteria.

In an alternative transdermal iontophoresis method, a desired depth of penetration for delivery of a drug is determined. Using criteria selected from the group consisting of i) AC frequency/pulse duration versus nerve or muscle tissue stimulation, ii) maximum current density to avoid skin burning, and iii) ionic concentration versus depth of penetration and AC frequency/pulse duration, an optimized AC frequency/pulse duration, current density, and ionic concentration for achieving the desired depth of penetration are determined. Finally, transdermal iontophoresis is carried out using the optimized AC frequency/pulse duration, current density, and ionic concentration.

In an improved transdermal iontophoresis system the electrical signal used to perform transdermal iontophoresis is an AC signal having a frequency of greater than about 100 Hz, and more preferably in the range of about 200-500 Hz. In an alternative transdermal iontophoresis system the electrical signal used to perform transdermal iontophoresis is an alternating signal having a pulse duration of less than about 2 milliseconds, and more preferably less than 1 millisecond. In a further improved transdermal iontophoresis system, the ionic concentration of the drug is varied to achieve a desired depth of penetration.

### BRIEF DESCRIPTION OF THE DRAWINGS

FIG. 1 is a block diagram of the transdermal iontophoresis system of the invention.

FIG. 2 is a graph illustrating the AC frequency threshold for nerve fiber stimulation.

FIG. 3 is a graph illustrating the current threshold for nerve fiber stimulation as a function of the duration of a square pulse applied to the nerve fiber.

FIGS. 4A-D are cross-sectional views (not to scale) showing the movement of the leading edge of a drug being administered using transdermal iontophoresis during a long pulse or long half-cycle of a low frequency AC signal, and comparing the system of the invention to prior systems.

FIG. 5 illustrates an array of electrodes according to the invention.

FIG. 6 illustrates a pair of interlaced arrays of electrodes according to the invention.

FIG. 7 is a cross-sectional view (not to scale) showing transdermal iontophoresis using a pair of electrode arrays.

FIG. 8 is a cross-sectional view (not to scale) showing transdermal iontophoresis using an electrode array.

FIG. 9 is a cross-sectional view (not to scale) showing transdermal iontophoresis using an electrode array whose elements are connected to a data bus.

FIG. 10 is a graph showing the relationship between electrode size, AC frequency, and depth of penetration.

FIGS. 11A and 11B illustrate alternative electrode assemblies in which pairs of individual electrodes are connected.

### DETAILED DESCRIPTION OF THE INVENTION

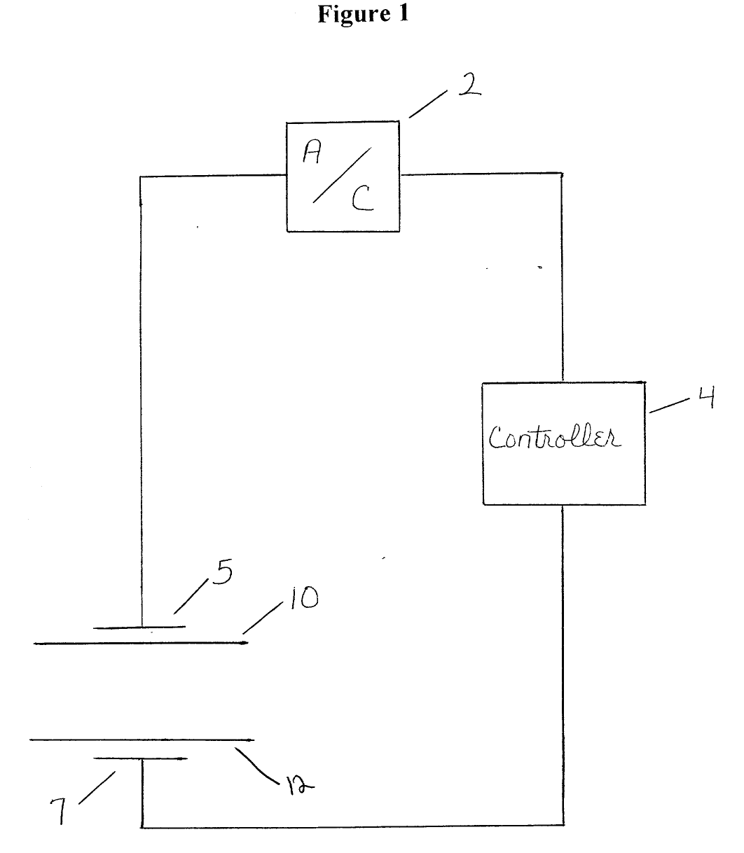

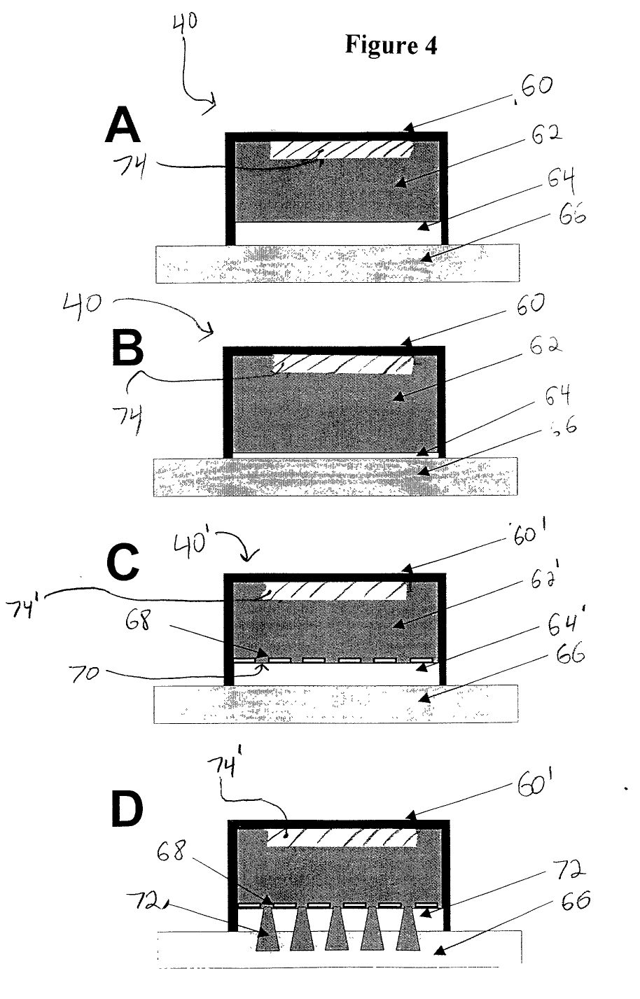

The present invention is a method of and apparatus for optimizing and controlling the rate and depth of delivery of a drug using transdermal iontophoresis while avoiding burning and other adverse effects normally associated with transdermal iontophoresis. Referring to FIG. 1, the system of the invention is similar to prior systems and includes a power source 2 controlled by a controller 4. Power source 2 generates an electrical current which is applied through electrodes 5, 7. The flow of current from one electrode or set of electrodes 5 to the other electrode or set of electrodes 7, and vice versa, carries ionized molecules of the drug through skin 66 (see FIG. 4) into the body.

As discussed above, the use of transdermal iontophoresis is known to induce muscle and nerve tissue stimulation, and to cause burning in the area in which the transdermal iontophoresis electrodes are mounted due to localized heat excess. In order to overcome the problem of muscle stimulation, the invention uses an electrical signal, and preferably an AC signal, with a frequency/pulse duration selected to enable high current intensity to be maintained while minimizing the stimulation of nerve and muscle fibers.

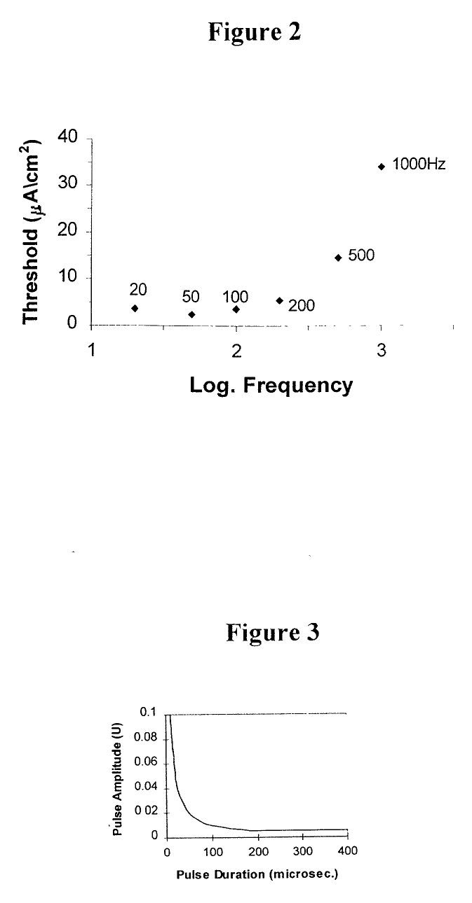

FIG. 2 is a graph showing the threshold at which nerve fiber stimulation occurs during transdermal iontophoresis. As shown, the threshold of nerve fiber stimulation increases as the AC frequency of the electrical signal used to perform transdermal iontophoresis is increased, i.e., as the pulse duration of the AC signal decreases. In particular, as the frequency of the electrical signal exceeds about 50 Hz, the electrical current density required to cause nerve stimulation increases exponentially. Thus, in the transdermal iontophoresis system of the invention, an AC signal frequency of preferably greater than 50 Hz is used. In a more preferred embodiment, the frequency of the AC signal is in the range of 100-500 Hz, and more particularly in the range of 200-500 Hz. Of course, frequencies above 500 Hz may be used as well, as shown in FIG. 2, with similar results with respect to nerve and muscle tissue stimulation.

Since the relationship between frequency of the electrical signal is directly related to the duration of the pulse of the signal, pulse duration has a direct relationship in reducing nerve stimulation as well. As shown in FIG. 3, the threshold for stimulating nerve fibers as a function of the duration of a square shaped pulse applied to the nerve increases exponentially as the length of the pulse decreases, especially at pulse durations of less than 2 milliseconds. Thus, for non-sinusoidal AC signals used in transdermal iontophoresis according to the invention, the pulse duration of the electrical signal used to administer the desired drug is controlled to have a pulse duration of less than 2 milliseconds, and more particularly less that 1 millisecond. Using these electrical criteria, high intensity current required for effective drug delivery may be applied without stimulating nerve and muscle fibers.

In a preferred embodiment, AC square-wave pulses or sine waves having the characteristics set forth above are used. The pulses may also be applied at different intervals, pulses may be of alternating polarity, and the pulse shapes may be exponential or in any desired configuration provided that the frequency/pulse duration is maintained at a level to minimize or prevent muscle stimulation.

Effective drug delivery is maintained even when a symmetric set of pulses (positive and negative of the same amplitude and duration) are used due to the asymmetric nature of the electrode, human tissue, body fluids, and combinations thereof. Near the electrodes, where the concentration of the drug is high, the drug constitutes the majority of the current conduction ions. Therefore, when current is passed between the electrodes, the fraction of current carried by the drug into the body is very high. This fraction, however, decreases with penetration depth as the drug concentration becomes lower relative to the concentration of other ions. When the current is reversed and the current begins to carry the drug back toward the electrode, the drug concentration is low within the body, and the majority of the fraction of current is carried by ions in the extracellular fluid (mostly sodium and chloride ions). Therefore, the drug accumulates in the body because only a fraction of the amount of the drug that migrated into the body during one half-cycle migrates out of the body during the next half-cycle despite the symmetric current and no net flow of current. The symmetric AC current has the added benefit of avoiding an accumulation of ions around the electrodes, which might otherwise cause irritation.

It is critical in a transdermal iontophoresis system to ensure that the drug being administered achieves a desired depth of penetration so that it migrates into the desired tissue, whether that tissue is shallow or deep. When a current flow commences during transdermal iontophoresis, the charged drug moves in the desired direction for the duration of the pulse. The envelope or area containing the drug will include a front edge having a low concentration of the drug with the concentration increasing toward the trailing edge of the envelope at the drug source. As discussed in greater detail below, the rate of propagation or velocity of the front edge is a function of current density and ionic concentration. When the duration of the pulse is too short, e.g., due to the use of a high frequency AC signal to deliver the drug, if the current density is not large enough the drug may not reach the targeted depth. On the other hand, too great a current density in order to generate penetration to a desired depth may generate intense heat to the extent where the skin would be burned.

In the present invention, different electrode configurations are used to achieve drug delivery to a desired penetration depth while not exceeding the average current density over the area covered by the electrode that would cause damage to the skin or other tissue. In these embodiments, each electrode is configured so that at any given time only a portion or portions of the area of the electrode are used to deliver the drug. Within the area of these portions, the current density may exceed the level that would normally cause burning. The invention takes advantage of the fact that skin is thermally conducting and has a high heat capacity. Thus, if the area of heat application is small, blood circulation carries the excess heat away without burning or tissue damage. By ensuring that the average current density over the total area of the electrode assembly does not exceed that required to cause burning, greater current density, whether an AC or DC signal is used, may be achieved in localized areas without burning. Moreover, by restricting the area of current application to discrete areas of conductivity within the electrode, the current streams are intensified to form “current jets” having a higher velocity of drug propagation.

FIGS. 4A and 4B show the configuration of a typical transdermal iontophoresis electrode, as contrasted to FIGS. 4C and 4D, which show an electrode according to the invention, which utilizes current jets. FIG. 4A shows a typical electrode assembly 40 which includes a housing 60 which defines an interior chamber 62 in which the drug to be administered is contained, and to which is mounted an electrically conducting electrode 74. Chamber 62 is sealed by a porous membrane 64, which is pressed against the skin 66 during use, either directly or in the presence of a substance to improve the electrical contact between the electrode assembly 40 and skin 66.

As shown for illustrative purposes in FIG. 4B, when an AC current with is applied to electrode 74 through a lead wire (not shown) with a current at a low enough average current density to prevent burning of the skin under the electrode, drug 62 begins to migrate through membrane 64, but does not penetrate membrane 64 into skin 66 before the polarity of the AC signal reverses.

Referring to FIG. 4C, in the present embodiment of the invention, electrode assembly 40′ includes a housing 60′ which defines an interior chamber 62′ in which the drug to be administered is contained, and an electrically conducting electrode 74′. Chamber 62′ is sealed by a porous membrane 64′ which is pressed against the skin 66′ during use. An insulating layer 68 having a plurality of apertures 70 extending therethrough is included between chamber 62′ and membrane 64′. As shown in FIG. 4D, when the current is applied to electrode 74′, the current will not be conducted through insulating layer 68, but is instead directed through apertures 70. The current, which is preferably at a constant current level, is directed through apertures 70 to form current jets 72. Since the current is at a constant level, the average current density within current jets 72 is much greater than in a conventional electrode, thereby increasing the penetration depth of the drug. Apertures 70 may be cut, molded or otherwise formed in insulating layer 68, and may have any desired shapes, e.g., round, square, etc. The density of the apertures, i.e., the ratio of the area of the apertures to the total area of the insulating layer, as well as the area of each individual aperture, may be adjusted, in combination with adjustment of the current applied to electrode 40′, to achieve a desired current density and/or a desired depth of penetration.

Insulating layer 68 prevents application of current to the skin over the total skin area under electrode 40′. Instead, current is applied to the skin only in discrete areas adjacent to apertures 70, whereby the total surface area of the skin conducting an electrical current is reduced, and the average current density over the area under electrode 40′ is reduce to below the threshold for causing burning. The localized heat generated near apertures 70, which might normally exceed the current density that would cause burning, is carried away by blood circulation, and the average exposure to heat under electrode 40′ does not exceed the threshold at which the skin will burn.

Current jets 72 also provide additional penetration depth. Because the current is being forced into smaller areas of conductivity, i.e., apertures 70, the current density and velocity of current (charges) flowing through apertures 70 increases. The velocity determines the depth of penetration, since at the end of the duration of the current pulse or of the AC half-cycle, the flow is stopped and the penetration stops. Therefore, the greater the velocity of the charges, the deeper they penetrate within the given time of flow. Consequently, using current jets not only minimizes burning, but also enables increased depth of penetration.

Control of depth of penetration is achieved by varying the size of apertures 70, which will enable control of both velocity (as discussed below) and current density, and by controlling the constant current applied to electrode 60′. The velocity of the current through apertures 70, and the resultant depth of penetration, may be increased by decreasing the size of the current jets.

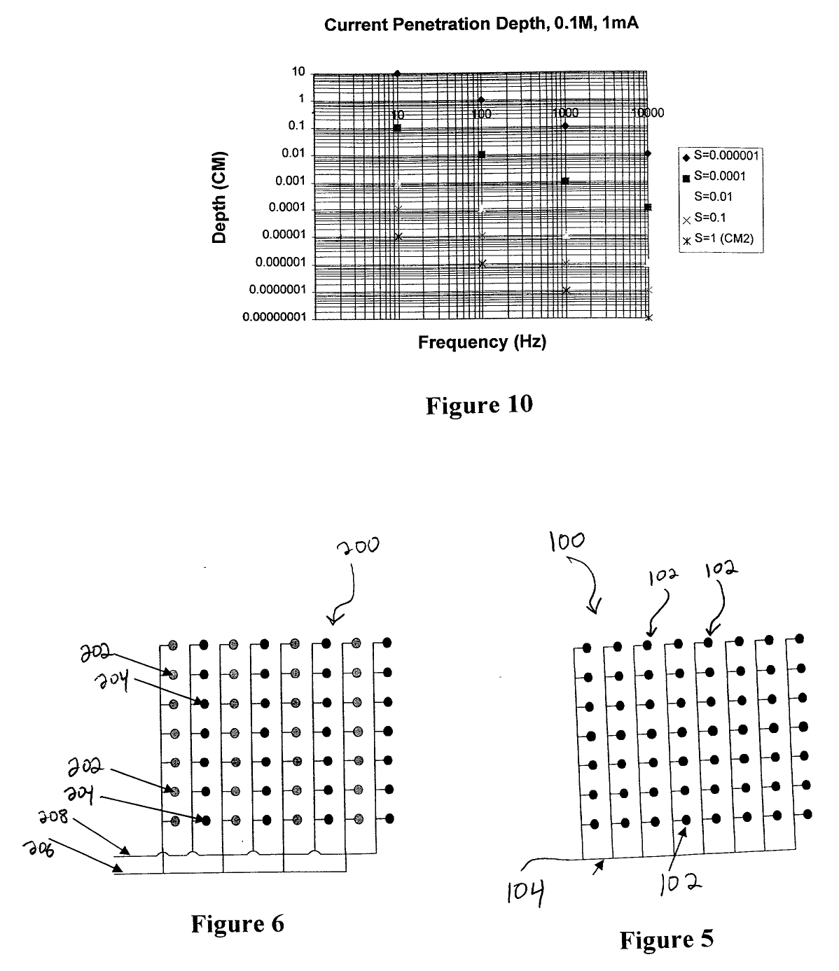

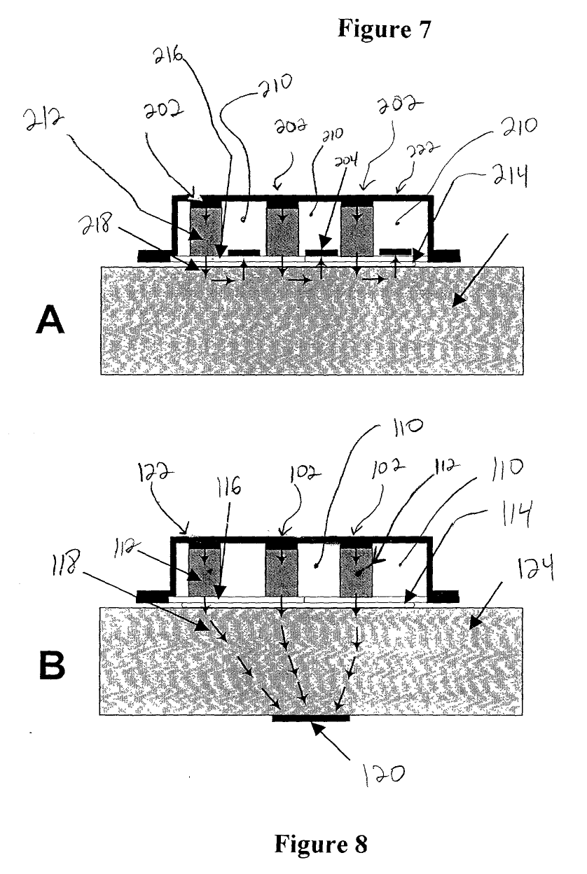

FIGS. 5-8 show alternative embodiments of the invention in which multiple electrodes are formed into one or more arrays for shallow drug penetration (FIGS. 6 and 7) and for deeper penetration (FIGS. 5 and 8). Referring to FIG. 5, electrode array 100 is formed of an array of electrodes 102. An electrical lead 104, is electrically connected to each electrode 102 for delivering an electrical signal through the electrodes 102. Each electrode may be constructed of any desired conducting material, such as metal, carbon sheet, silicon etc. Each electrode may also have any desired shape, e.g., round, square, etc., and the individual electrodes need not be the same size, although that is preferred.

Referring to FIG. 8, electrodes 102 are preferably mounted on a non-conducting base 122, which forms a housing for the electrode. Base 122 is preferably constructed of a plastic, such as polystyrene or polyethylene, or of rubber, silicon, silicon rubber, or any other non-conductive material. Within the housing defined by base 122, compartments 112 in which the drug to be delivered is contained, are formed by barrier walls 110. Barrier walls 110 are preferably constructed of an electrically non-conducting material, such as a plastic, and may be integrally molded with base 122. A membrane 116 of the type utilized in conventional transdermal iontophoresis electrodes forms a boundary between the drug in chambers 112 and skin 114. Barrier walls 110 extend on all sides of each electrode 102 in order to electrically isolate each electrode, and the drug in the compartment associated with such electrode, from the other electrodes and compartments.

Electrodes 102 are mounted to base 122 using any conventional means, such as adhesive, or base 122 may be molded around electrodes 102. In use, an electrical current is communicated to electrodes 102 through lead 104 (not shown in FIG. 8). The current flows through electrodes 102 directly through the drug in chambers 112, though membrane 116 and skin 114, and into the body 124. A corresponding electrode 120 receives the flow of current from electrode array 100. In this embodiment, the drug is induced into the body only during one half-cycle of the AC signal, i.e. the half-cycle in which current is flowing from electrodes 102 toward electrode 120. Pairs of electrodes of this type may be used to deliver a drug during the full AC cycle. Barrier walls 110 prevent the current from flowing in any direct other than interior of the body, and with the current flow only in discrete areas, current jets 116 are formed, which have a greater current density and higher current velocity, as discussed above. Furthermore, while the localized heat density near current jets 116 may exceed a normally desired threshold level, the average current density over the area of the electrode does not exceed this threshold and the excess local heat is dissipated by blood circulation.

In order to vary the current density (and therefore the depth of penetration), the spacing between barriers 110 may be adjusted as desired. Also, electrodes 102 and 120 may be spaced, so that the current flow must traverse a greater distance to providing deeper penetration. As discussed in more detail below, the size of electrodes 102 may be adjusted as desired to achieve a desired depth of penetration.

In an alternative embodiment of the invention, as shown in FIGS. 6 and 7, multiple transdermal iontophoresis electrodes are formed into an interlaced electrode array assembly 200. Referring to FIG. 6, electrode assembly 200 is formed of a first array of electrodes 202 interlaced with but electrically insulated from a second array of electrodes 204. Electrical lead 206 electrically connects to each electrode 202 for delivering an electrical signal to electrodes 202, and electrical lead 208 electrically connects to each electrode 204 for delivering an electrical signal thereto. Each electrode 202, 204 may be constructed of any desired electrically-conducting material, and may have any desired shape and size.

Referring to FIG. 7, electrodes 202 are preferably mounted on a non-conducting base 222, which forms a housing for the electrode assembly, and which is preferably constructed of a plastic. Within housing 222, compartments 212 defined by barrier walls 210 store the drug to be administered. Mounted to barrier walls 210 are electrodes 204, which are preferably molded directly into barrier walls 210 or are mounted with adhesive or other fastening means. A membrane 216 forms a boundary between the drug in chambers 212 and skin 214, and is preferably in touching or other electrical contact with electrodes 204. Barrier walls 210 extend on all sides of each electrode 202, in order to electrically isolate each electrode 202, and preferably extend along the sides of electrodes 204 to prevent a short-circuit between the drug in chambers 212 and electrodes 204.

In use, an AC electrical current is communicated to electrodes 202 and 204 through leads 206 and 208 (not shown in FIG. 7). The current flows through electrodes 202, through the drug in chambers 212, though membrane 216 and skin 214, into the body 224, and out of the body into electrodes 204. During the reverse cycle of the AC signal, current flows through the same path in the opposite direction. Barrier walls 210 are preferably shaped to minimize the likelihood of short-circuiting developing between the drug in chambers 212 and electrodes 204 in order to ensure maximum drug delivery into the body. The schematic current flow lines 218 where the drug is carried by the current show that the distance traversed by the current is small due to the close proximity of electrodes 202 and 204. By varying the distance between electrode 202 and 204, e.g., by increasing the size of barriers 210, it is possible to further control penetration of the drug. If desired, each electrode 204 may include an associated chamber 212 containing the drug, similar or identical to the chambers associated with electrodes 202. In this manner, drug delivery may be effectuated through the full AC cycle.

In the embodiments shown in FIGS. 7 and 8, chambers 212 and 112, respectively, are sized so that only a fraction of the total surface of the electrode assembly is available for current conduction. If desired, the drug in the drug chambers may be fed from a larger chamber (not shown) used to store the drug, with smaller pathways enabling the drug to flow to chambers 112, 212.

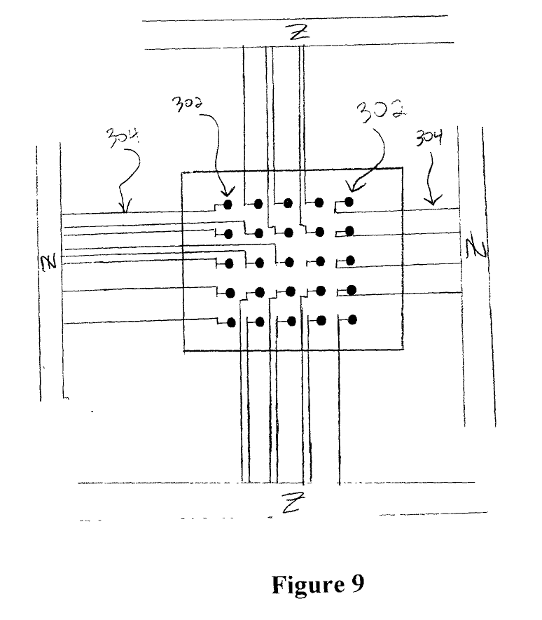

FIG. 9 shows a further alternative embodiment, in which each electrode 302 receives an electrical signal from its own associated electrical lead 304. In this embodiment, each of the leads is connected to a controller (not shown) by means of a data bus Z or by direct connection. The controller, may be, for example, a multiplexor in which each electrode or group of electrodes is individually selected and the electrical signal applied therethrough. The electrodes may be scanned, whether randomly, sequentially, or in any desired pattern to generate an electrical signal through the electrodes in the desired scanning pattern.

A further alternative embodiment of the invention is shown in FIGS. 12A and 12B. Referring to FIG. 12A, electrode assemblies 410 and 412 include grouped pairs of individual electrodes, e.g., electrodes 400A and 400B, 402A and 402B, etc., which are connected together by individually addressable lead lines. Each pair of electrodes may be individually activated by a controller (not shown) to cause a transdermal iontophoresis electrical signal to flow between the individual electrodes of the selected pair. For example, the controller may cause the electrical signal to flow between electrodes 400A and 400B, and then between electrodes 402A and 402B. In order to control the electrode pairs, the controller may multiplex between the electrode pairs, or may, for example, induce the desired electrical signal in a secondary coil between the electrode pairs by activating an electrical signal through a desired primary coil associated with each electrode pair. Of course, more than one electrode pair may be activated at a given time, and the connections between individual electrodes may configured so as to allow the electrode pairings to provide a desired delivery pattern.

FIG. 12B is similar to the embodiment shown in FIG. 12A but in a single electrode assembly configuration. Electrode assembly 416 includes electrode pairs, e.g., electrodes 418A and 418B, with a lead line connecting each electrode of the pair. Each electrode pair may be individually addressed by a controller (not shown) to cause the transdermal iontophoresis electrical signal to flow between the selected electrodes. If desired, the depth of penetration may be controlled by configuring the electrode assembly so that the pairs are farther spaced, e.g., electrodes 418A and 418B are paired and electrodes 420A and 420B are paired) for deeper penetration, and are more closely spaced, e.g., electrodes 418A and 420A are paired and electrodes 418B and 420B are paired) for shallower penetration. The electrodes may also be paired in any other desired configuration, and may be addressed by the controller, to achieve any desired pattern for delivery of the transdermal iontophoresis electrical signal.

Electrodes 102, 202, 204, 302 may be constructed in any desired shape, e.g., rectangular or circular, and are preferably sized in the range of 0.1 to 1 mm2, with the total area of all electrodes in an electrode assembly on the order of 1-10 cm2. The distance between the individual electrodes is preferably in the range of 0.1 to 1.0 mm, although variation is foreseen with respect to each of these parameters. Thus, for each electrode assembly, the total number of electrodes may be in the thousands, although far smaller electrode arrays are foreseen. Such electrode arrays, including the connections to the individual electrodes may be fabricated using microelectronics fabrication techniques, if desired. For example, the electrodes may be silicon, with insulating layers of the type known in the art separating the individual electrodes. Printed circuit technology may be also employed, if desired.

Various alternative embodiments are foreseen with respect to the electrode arrays disclosed. These alternatives may be used to increase or decrease the depth of penetration as desired. For example, if deeper penetration is desired, the electrode or individual electrode(s) activated at a given time may be farther spaced. An increased distance between active electrodes, i.e., electrodes for which an electrical signal is applied thereto at a given time, increases the current jet effect and results in deeper penetration. Also, by adjusting the size of the individual electrodes of the electrode array, the depth of penetration may be controlled. FIG. 10 shows an example of this relationship for the case of a 0.1 Molar concentration solution of a drug at a constant current of 1 mA, where S is the area of an individual electrode measured in cm2. As shown, depth of penetration increases linearly with a decrease in electrode size at a constant frequency. Thus, electrode size may be adjusted to achieve a desired penetration depth.

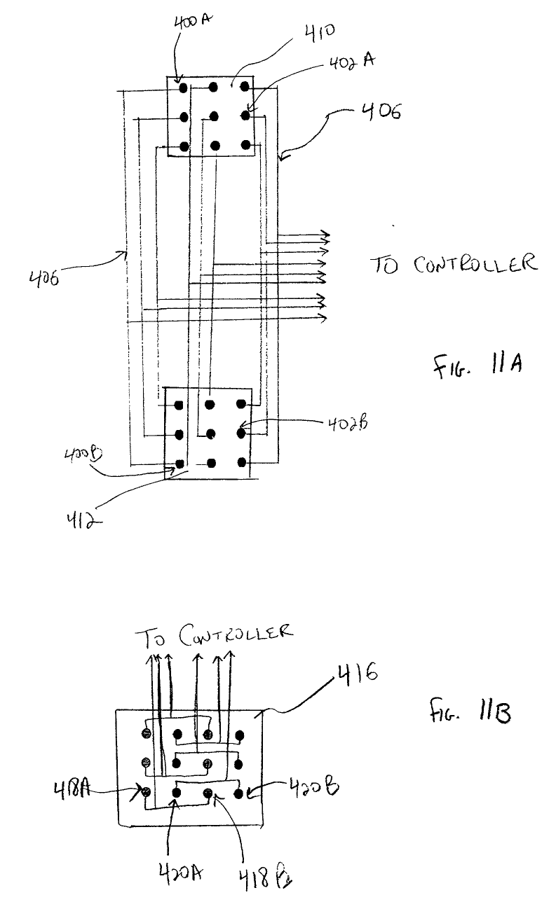

In order to further control the penetration depth of the drug, the ion concentration of the drug may be varied. This has the effect of controlling the velocity of the drug when subject to an electric current. In general, the drug concentration should be as high as possible relative to other charge carriers. However, the total ion concentration should be kept low if deep penetration is required, and should be made higher if shallower penetration is desired. This is because, as shown in FIG. 11, penetration depth is inversely proportional to ionic concentration at a given current. This relationship exists because a charge carrier's velocity at a given current, i.e., a given number of charges traversing any cross-section of flow path per second (coul/sec), is dependent on the number of ions present to conduct the electrons. Since the charges must travel from one electrode to another, if the number of conducting ions per conducting volume (density) is low, a faster more efficient, path must be taken during the duration of the AC pulse in order to accommodate the number of charges that must travel by means of the current. Hence, the overall velocity of each charge traveling in the current must increase in order to minimize queuing. As illustrated in FIG. 11, at a given current, the depth of penetration is halved if the ionic concentration is doubled. Thus, in order to control penetration depth, the ionic concentration should be adjusted and, to the extent possible, the drug should contain as many charges as possible relative to the carrier in which the drug is dissolved, without exceeding the total ionic concentration desired, in order to maximize the drug delivery at the ionic concentration.

To properly deliver drugs by means of the present invention, the specific type of delivery has to be taken into account. For delivery to the superficial skin layer, e.g., for local anesthesia of the outer skin during cosmetic laser treatment, less depth of penetration is required, whereas for systemic drug delivery, high drug penetration is required. Also the former requires relatively small drug doses (very little dilution in a very small volume) that will rapidly (within seconds) and transiently (duration of minutes) provide local anesthesia, while the latter usually calls for slow, long term (hours) delivery of sufficient doses that will be effective when diluted in the entire body fluid volume. Other factors, such as burning pain threshold and muscle stimulation threshold must be taken into account as well. On the basis of these factors, the current density, the wave frequency or pulse duration and repetition rate, as well as the ionic concentration of the drug solution are selected. The selection further involves the selection of the appropriate electrodes. For example, the deeper the required penetration, the smaller the area of the individual electrodes and the longer the pulse duration should be selected. Alternatively, electrodes with large total active surface areas (closely packed electrodes) may be selected when superficial penetration is required, for example for local anesthesia of the skin, cosmetic treatments, or large doses of the drug are to be administered, or in cases where the drug is ineffective as a charge carrier. For low penetration depth applications of the systems, e.g., skin anesthesia, in which there is a minimal danger of nerve-muscle stimulation and/or charge accumulation, it is foreseen that uni-directional pulses may be used.

Power source 2 may be any constant-current source of electrical power capable of delivering the desired electrical signals. Such power sources are well-known in the art. Any analog, digital or other current source with the appropriate current shaping capability, waveform generation, complex pulse shape and interval generation, etc. may be used. The end stage of the current source should be of a constant current type with an output consistent with that of conventional transdermal iontophoresis systems.

Controller 4 may be of any type of controller known in the art, such as a microprocessor with appropriate feedback control sensors. Automatic or manual controls (not shown) may be provided for enabling adjustments to the electrical signal generated by the system, e.g., to vary the AC frequency/pulse duration and current level, to control the depth of penetration as desired. The rate of drug delivery is controlled by the controller by adjusting the current amplitude, and pulse duration and intervals or waveform frequency. A provision for adding an asymmetry to the net current (by a bias current or using asymmetric pulses) in order to overcome rectification and other biological factors, may be used as well.

In order to assist in the selection of desired depth penetration within the comfort level of the patient, the factors for selecting the desired delivery criteria, i.e., frequency or pulse duration, electrode configuration (including the use of electrodes with different electrode surface area and/or distance between electrodes), and current density, etc. may be manually controlled. In a preferred embodiment, a microprocessor and software, or other controller, is programmed to automatically calculate the various factors based on user-entered information, e.g., drug delivery rate, depth of penetration, or magnitude of drug to be administered, etc. Feedback sensors may be used to vary frequency, pulse duration, current density etc., for automatic control. For example, controller 4 preferably maintains the desired electrical parameters despite changes in the system, such as impedance changes etc. One or more sensors may also be used to measure the concentration of the drug being delivered. The sensors may be local, e.g., closely adjacent to the electrodes being used for drug delivery, or may be systemic, measuring system-wide concentration of the drug, or a reaction to the drug indicating the systemic presence of the drug, e.g., for detecting insulin delivery, control may occur based upon blood glucose levels. Sensors may also be provided to measure the temperature of the skin adjacent to the electrodes. This temperature value may be communicated to controller 4 and used as a parameter to determining current density and other parameters controllable in the system.

The system of the invention may be used in combination with other known techniques known in the field of transdermal iontophoresis. For example, the system may be used in combination with techniques known to lower the resistance of the skin and surrounding tissue during transdermal iontophoresis, such as described in U.S. Pat. No. 5,622,168. If a current conductive paste (not shown) is positioned between the electrodes and the skin, the thickness of the paste should be minimized, preferably to less than 0.01 mm, in instances where depth of penetration due to the operational parameters selected, e.g., an AC signal of relatively short pulse duration, is of concern.

Although the present invention has been described with respect to certain embodiments and examples, alternatives exist that will be appreciated by those skilled in the art and that are within the scope of the invention as described in the following claims.

1. A transdermal iontophoresis electrode comprising:
a housing having an interior and an open end extending from the interior of the housing to an exterior of the housing;
an electrode disposed within the housing; and
an insulating layer disposed adjacent to the open end of the housing, the insulating layer comprising at least one aperture extending therethrough, the at least one aperture having a cross-sectional area less than the cross-sectional area of the open end of the housing.

2. The transdermal iontophoresis electrode according to claim 1 wherein the size of the aperture is selected to achieve a desired depth of penetration.

3. The transdermal iontophoresis electrode according to claim 1 further comprising a plurality of apertures extending through the insulting layer, the combined cross-sectional area of the apertures being less than the cross-sectional area of the open end of the housing.

4. The transdermal iontophoresis electrode according to claim 3 wherein the size of each aperture is selected to achieve a desired depth of penetration.

5. The transdermal iontophoresis electrode according to claim 1 further comprising a membrane adjacent to the open end of housing.

6. The transdermal iontophoresis electrode according to claim 1 further comprising at least one dividing member within the housing defining a plurality of channels within the housing, each channel having an interior and an open end extending from the interior of the channel to an exterior of the channel; and
the insulating layer comprising at least one aperture adjacent to the open end of at least one channel, the aperture having a cross-sectional area less than the cross-sectional area of the open end of the at least one channel.

7. The transdermal iontophoresis electrode according to claim 6 wherein the insulating layer comprises at least one aperture adjacent to the open end of each channel, each aperture having a cross-sectional area less than the cross-sectional area of the open end of its respective channel.

8. A transdermal iontophoresis electrode assembly comprising:
a housing;
at least one dividing member within the housing defining a plurality of channels within the housing, each channel having an interior and an open end extending from the interior of the channel to an exterior of the channel; and
an insulating layer adjacent to the open end of the housing, the insulating layer comprising at least one aperture adjacent to the open end of at least one channel, the aperture having a cross-sectional area less than the cross-sectional area of the open end of the at least one channel.

9. The transdermal iontophoresis electrode according to claim 7 wherein the insulating layer comprises at least one aperture adjacent to the open end of each channel, each aperture having a cross-sectional area less than the cross-sectional area of the open end of its respective channel.

10. The transdermal iontophoresis electrode according to claim 8 wherein the size of the aperture is selected to achieve a desired depth of penetration.

11. A transdermal iontophoresis electrode comprising a housing having an interior and an open end extending from the interior of the housing to an exterior of the housing, the interior having a cross-section, the open end comprising an aperture having a cross-section smaller than the cross-section of the interior.

12. The transdermal iontophoresis electrode according to claim 11 wherein the size of the aperture is selected to achieve a desired depth of penetration.
